(19) United States
(12) Patent Application Publication
(10) Pub. No.: US 2011/0028776 A1
(43) Pub. Date: Feb. 3, 2011

(54) Packaged Magnetic Therapeutic Topical Preparation

(76) Inventor: Donald Spector, New York, NY (US)

Correspondence Address: COLLARD & ROE, P.C., 1077 NORTHERN BOULEVARD, ROSLYN, NY, 11576, US

(21) Appl. No.: 12/902,732

(22) Filed: Oct. 12, 2010

Related U.S. Application Data
(63) Continuation-in-part of application No. 11/355,331, filed on Feb. 16, 2006, now Pat. No. 7,850,591.
(60) Provisional application No. 60/771,078, filed on Feb. 6, 2006.

(51) Int. Cl. A61N 2/08; A61M 35/00

(52) U.S. Cl. 600/9; 604/289

(57) ABSTRACT

A topical preparation externally applied to the skin, the preparation containing particles that can be magnetized. The preparation being stored in a container with a magnetic source.

This application is a continuation-in-part of U.S. patent application Ser. No. 11/355,331, filed Feb. 16, 2006, which claimed priority from U.S. Provisional Application No. 60/771,078, filed Feb. 6, 2006. All of the foregoing applications are hereby incorporated herein by reference in their entirety.

### BACKGROUND ART

Exposure of organs, bones and tissue to static magnetic fields provides relief to patients suffering pain. A number of articles have addressed the therapeutic qualities associated with magnetic fields. A number of products have been designed, most of which do not work well. They do not work well, in part, because they do not apply a magnetic field directly to the area that needs treatment.

New and improved methods and apparatus to apply magnetic fields to objects to obtain a therapeutically beneficial result are required.

### SUMMARY OF THE INVENTION

In accordance with an embodiment of the present invention, a topical preparation containing magnetizable material is packaged in a container having a magnetic source. The magnetizable material may be ferrous particles mixed into the preparation. The preparation is of a viscosity sufficient to maintain the particles mixed within the preparation over the pull or push of the magnetic source. The preparation is thus, already magnetized when applied to a patient for use. After applying the preparation to the skin of a patient, the container and its magnetic source can later be brought near to the skin to recharge the magnetic field given off by the magnetizable material. The magnetic source may be a permanent magnet or an electromagnet.

In accordance with the teachings herein, a device that is a magnetizer is presented. The magnetizer, in accordance with another aspect of the teachings includes a meter. This will disclose, with some degree of accuracy, the magnetic charge that it has emitted to either internal screws, orthopedic devices or micro encapsulated ferrous substances. This meter is easy to use and inexpensive, however since magnetic materials discharge over time it is important to be able to measure, control and replace the fading magnetic charge as needed. The meter is used in conjunction with the magnetic field system, but does not necessarily have to be connected, since the patient may wish to monitor the field at home. Thus, the meter and the magnetizer can be provided in kit form. The magnetizer can be used on micro encapsulated topical preparations that includes magnetic particles. The magnetism can also be used to increase the charges of metallic, ferrous or other magnetizable object already inside the body.

The magnetizer, preferably has the proper warning for people with pacemakers or other devices that might be sensitive to magnetic fields.

In accordance with a further teaching described herein, an apparatus for magnetizing materials for therapeutic purposes includes a magnetic field generator that generates a magnetic field and a meter that measures magnetic field strength. The apparatus preferably, but not necessarily, includes a switch that enables and disables the operation of the magnetic field generator. The switch can also enable and disable the meter, further preferably disabling the meter when the magnetic field generator is enabled and enabling the meter when the magnetic field generator is disabled. Alternatively, as a second switch that enables and disables the meter can be provided.

In accordance with another teaching described herein, the meter can be enabled when the magnetic field generator is enabled to measure the magnetic field generated by the magnetic field generator.

A control that regulates the strength of the magnetic field can also be provided on the apparatus. A timer that enables the magnetic field generator for a selected time can also be provided on the apparatus.

In accordance with a further teaching described herein, the apparatus is used to apply a dosage of the magnetic field to an object in a human body so that the object generates a therapeutic magnetic field. The object can be any magnetizable object, including but not limited to orthopedic devices. Such orthopedic devices can include metallic screws and pins.

In accordance with another teaching described herein, the apparatus is used to apply a magnetic field to a pill so that the pill generates a therapeutic magnetic field. The pill can be, for example, a microencapsulated metallic material or a microencapsulated ferrous material. The pill also includes a therapeutic amount of a drug or other material. The apparatus is used to apply a magnetic field to the pill when the pill is outside a body, and then the pill is swallowed. The meter can be used to determine the magnetic field emanating from the pill before or after swallowing.

In accordance with a further teaching described herein, the meter measures the therapeutic magnetic field on the object. The meter can also be used to measure magnetic fields associated with different parts of a body.

The meter can include a display. The display indicates a strength of a magnetic field emanating from an object in a human body. In accordance with one teaching, the display has an indication of whether the magnetic field is above or below a threshold. If the measured magnetic field is below a threshold, then the meter indicates by an alarm that the magnetic field generator should be used to apply a magnetic field to the object.

Also contemplated is a kit for magnetizing materials for therapeutic purposes. The kit includes at least two components, including a magnetic field generator that generates a magnetic field and a meter that measures magnetic field strength that is separate from the magnetic field generator.

The magnetic field generator and the meter can be used in the same manner previously described.

Also contemplated is a method of providing a therapeutic magnetic field. The method includes applying a dosage of magnetic field generated by a magnetic field generator to an object and measuring the magnetic field strength emanating from the object with a meter.

The strength of the applied magnetic field can be controlled by a controller or a timer. As previously described, the device can be an object inside a body or a pill.

The method can further include the step of applying a second dosage of magnetic field generated by the magnetic field generator if the magnetic field strength is less than a threshold.

In an alternative method, a meter can be used to measure the magnetic field emanating from an object before applying a magnetic field to the object. The magnetic field would be generated and applied to the object depending on the reading on the meter.

### BRIEF DESCRIPTION OF THE DRAWINGS

The foregoing features of the invention will be more readily understood by reference to the following detailed description, taken with reference to the accompanying drawings, in which:

FIG. 1 provides a diagram of a human body with illustrative examples of locations of magnetizable objects.

FIGS. 2 and 3 show magnetizing devices.

FIG. 4 illustrates a circuit of one aspect of a magnetizing device.

FIG. 5 shows a display on a meter on the magnetizing device.

FIG. 6 illustrates the use of one aspect of the present invention.

FIG. 7 illustrates the use of a magnetic field meter.

FIG. 8 illustrates a pill in accordance with one teaching described herein.

FIG. 9 illustrates a kit in accordance with one teaching described herein.

FIG. 10A is a side cross-sectional view of a magnetic topical preparation in a jar of an embodiment of the present invention.

FIG. 10B is a bottom view of a cap for the jar of FIG. 10A.

FIG. 11A is a perspective view of a tube of magnetic topical preparation according to an embodiment of the present invention.

FIG. 11B is a side cross-sectional view of a cap for the tube of FIG. 11A.

FIG. 12A is a perspective view of a magnetic topical preparation in a jar of an alternate embodiment of the present invention.

FIG. 12B is a side cross-sectional view of the jar of FIG. 12A.

### DETAILED DESCRIPTION OF SPECIFIC EMBODIMENTS

The present invention relates to the field of magnetic therapy. Many people experience beneficial effects (including a reduction in the feeling of pain) by having tissue, bones or organs being exposed to static magnetic fields. These fields are typically applied by magnets located outside the body.

A magnetic field generator and a meter provides dosages of therapeutic magnetic fields to patients. Advantageously, a kit that includes a magnetic field generator and a meter may be packaged together. These devices are used to magnetize objects that are inside a body, on the skin of a body or to be swallowed by a body so that the objects can provide a magnetic field. The magnetic field will be of greater strength because the objects are inside the body and will be of greater benefit because they are near tissue, organs or bones that require therapy to assist in healing. The meter can be used to measure the magnetic field on the object to allow the application of appropriate dosages. The meter can also be used to measure the magnetic field output by the magnetic field generator to allow accurate measurement of the dose of magnetic field applied.

In many cases patients already have objects or materials in their bodies, on their bodies or that will be put inside their bodies that can be magnetized. Metal parts like screws, fixators, plates, prostheses are examples of metal parts and implants that may be present in a body.

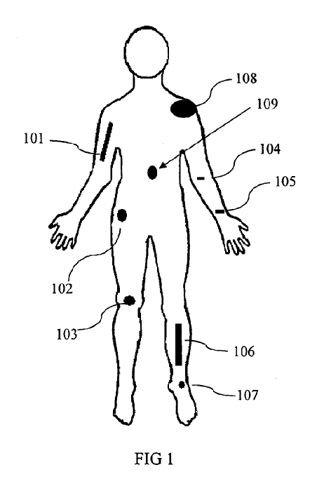

This is shown in diagram in FIG. 1. FIG. 1 is a diagram of the human body with, for illustrative purposes only, locations where metal parts and implants are known to be applied. This includes areas and items such as humeral implants 101, hip implants 102, knee implants 103, elbow joint implants 104, screws for joint fixation 105, tibial component of knee replacement 106 and implants for ankle reconstruction 107.

Other components that can be magnetized are pills that include a therapeutic material and a magnetizable material. For example, the pills may be micro encapsulated metallic or ferrous substances. These substances can be ingested, or implanted in the body. Referring to FIG. 1, a pill is illustrated in the digestive system 109 of the body.

Creams or lotions that include magnetizable material, such as metals, that can be applied to the exterior of the body, such as the skin at location 108. The magnetic material in the creams or lotions can also be magnetized so that the creams or lotions emit a magnetic field. For example, the creams or lotions can include any metal, such as ferrous material.

The apparatus or kit described above, either with or without the meter, applies a magnetic field to any of the objects illustrated in FIG. 1 to magnetize the objects. The objects will therefore emit a magnetic field. This magnetic field will be close to tissue, organs or bones that will benefit from the therapeutic effects of a magnetic field.

In an initial state, the magnetizable materials or objects may contain no, or a very weak magnetic field. It is also known that over time magnetized materials may lose strength in their magnetic field. In accordance with one approach, the strength of a magnetic field applied to the objects of FIG. 1 is determined so that the strength of the magnetic field emanated by the objects is maintained at a level that will not be reduced below a minimal desired strength. It is also preferable to magnetize the objects within a body to a defined level. It is also preferred to repeat magnetization of objects at a certain time with the same magnetic field as at previous occasions.

These needs are addressed by applying known and repeatable levels of magnetic field strengths. The device that is used for generating a magnetic field is a magnetic field generator. Magnetic Field Generators are well known. It is advantageous to provide a magnetic field generator as a therapeutic instrument that includes a meter to disclose the strength or the dosage of the magnetic field that is applied to the objects in the body.

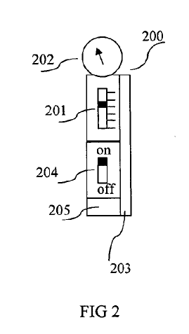

One embodiment of a therapeutic device is shown in FIG. 2. The device can be fixed in a rigid package. The device is preferably provided in a wand like construction and is completely mobile.

The magnetizing device 200 in FIG. 2 is comprised of the following components. It has a magnetic field generator 203 that generates a magnetic field. There are different known ways to generate a magnetic field, of which one is by electrically energized coils. The magnetizing device 200 also has a regulator 201. This regulator 201 allows the strength of the generated magnetic field to be controlled. In the case of an electrical magnetizer, the regulator may be a potentiometer that regulates the strength of the current to the coils and thus can regulate the strength of the magnetic field. The regulator 201 has different setting positions, which can be indicated by a scale. The magnetizer also comprises an on/off switch 204. The on/off switch enables and disables the generation of a magnetic field by the magnetic field generator 203 as desired. The magnetizing device 200 includes a meter 202. This meter can provide an indication of the strength of the generated magnetic field in the direct neighborhood of the magnetizer 200. Consequently, this specific magnetizer provides a method to generate a therapeutic magnetic field of known strength. And it can be used at different times, after having been used at different settings in a desired setting generating a magnetic field of known strength. In case the magnetizer is an electrical magnetizer, a power source 205 is provided.

The meter 202 can also be used to measure the magnetic field emanating from an object that has been magnetized. For example, the magnetic field strength emanating from any of the objects of FIG. 1. can be measured using the meter 202, however, the magnetic field generator 203 should be disabled when taking the measurement. Thus, according to one approach, the switch 204 enables the generator 203 and disables the meter 202 in one position and enables the meter 202 and disables the generator 203 in another position.

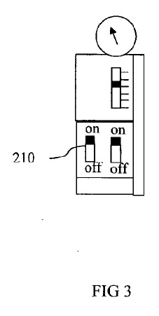

In FIG. 3, the magnetizing device includes a second on/off switch 210. The first switch is used to control the magnetic field generator 203 and the switch 210 is used to control the meter 202.

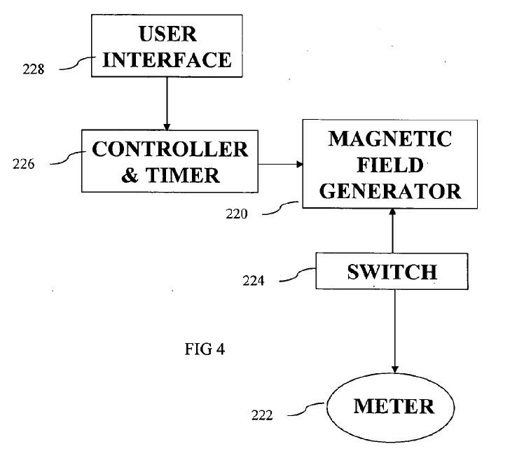

FIG. 4 illustrates a circuit diagram of the magnetizing device 200 of FIG. 2. The magnetic field generator 220 and the meter 222 are controlled by a switching circuit 224 in a manner already described. A controller and timer circuit 226 that is controlled by a user interface 228 controls the operation of the magnetic field generator 220. The controller and timer circuit 226 includes the regulator that regulates the strength of the output of the magnetic field generator 220. The regulator is controlled by a switch provided in the user interface 228. The controller and timer circuit 226 also includes a timer that controls the amount of time that the magnetic field is generated by the magnetic field generator 220. The timer is controlled by an interface provided in the user interface 228.

The meter 203 preferably has a display 240, shown in FIG. 5. A scale 242 is provided on the display 240. A threshold indicator 244 is provided on the scale. The position of the threshold indicator 244 on the scale 242 is preferably adjustable. A marker 246 is provided on the scale, the position of the marker 246 indicating the measured magnetic field strength. Two labels are preferably provided on the display 240. One label 248 is below the threshold indicator 244 and indicates that if the magnetic field measured on an object is less than the level of the threshold indicator 244, then an additional dosage of magnetic field should be applied to the object. The second label 250 is above the threshold indicator 244 and it indicates that no additional dosage of magnetic field should be applied.

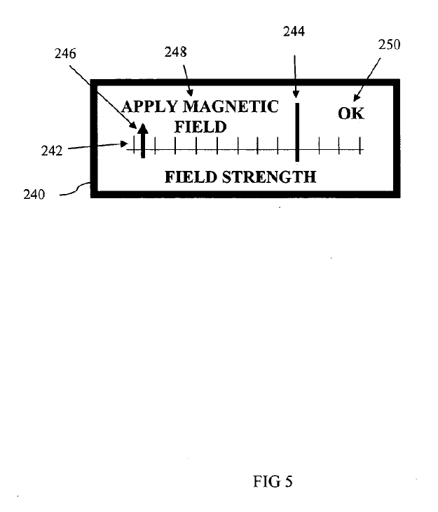

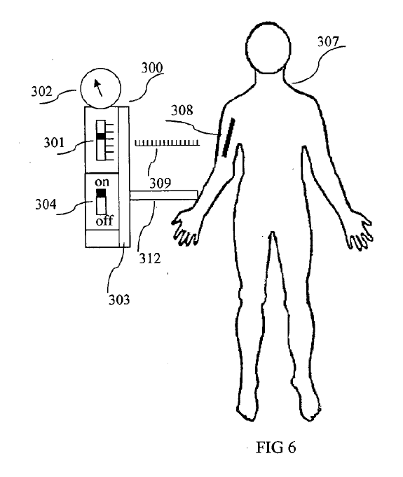

As an illustrative example, FIG. 6 shows how a magnetizing device 300 can be used on a patient 307 to magnetize an object 308 in the patient 307. The magnetizing device 300 is positioned in a known position by using, for example, a measuring tape 309. This allows the object 308 to be magnetized at different times with an identical magnetic field strength. The magnetizer has initially its switch 304 in the off position so that no magnetic field is generated. The regulator 301 is set in the desired position. If a timer is to be used, the length of time is selected through a user interface. The magnetizing device 300 is put in the desired position, if needed by using a device like the measuring tape 309. The switch 304 is put in the ON position. One can check on meter 302 if the magnetic field is of desired strength and if needed adjust the strength by regulator 301. Then, the magnetic field generator is disabled by a switch, and, the magnetic field strength emanating from the object 307 can be measured with the meter 302. For accurate readings, the device 300 is put into a known position with, for example, a tape measure. It is noted, however, that the use of a tape measure is entirely optional. Further, a measuring component 312 that extends from the device 300 can be provided to accurately measure the distance of the device 300 from a body 307.

It is known that the strength of a magnetic field that is measured on a distance from a magnetizer is not identical to the strength of the magnetic field close to the magnetizer. Also the magnetic field induced in an object or in micro encapsulated ferrous substances in a body may be different from the strength of a field generated by the magnetizer at the position of the object or the micro encapsulated substance. Further more it is known that materials may demagnetize over time and lose in strength of magnetic field over time. It is important to be able to measure, control and replace the fading magnetic field as needed. It is useful to provide a meter that can measure the strength of the magnetic field generated by objects or particles in the body. In this case, the meter is used in conjunction with the magnetic field system, but does not necessarily have to be connected to the magnetizer. Thus, the magnetic field generator and the meter can be provided in a kit as separate components. It may be desirable to measure or to monitor the strength of the magnetic field of objects and particles in patients away from the location of the magnetizer, for instance at home. Such meters can be inexpensive, simple and easy to use. They can for instance use available Hall Effect based magnetic field instruments.

The meter can also be used to measure the output of the magnetic field generator. The strength of the magnetic field measured from the generator may be significantly different than the strength of the magnetic field generated by an object in the body. Thus, the meter can have two selectable scales. One scale is selected when measuring an object and the second scale is selected if measuring the output of the magnetic field generator.

FIG. 7 shows how a meter can be used. A patient 401 has an object 402 that has been magnetized to a desired strength of magnetic field. The strength of the magnetic field has been measured directly after magnetization by a magnetic field meter 403. The position of this meter relative to the position of the magnetized object 402 in the patient has been recorded. It may have been measured by a measuring tape 404. It may also have been placed at a known position at the skin of the patient. At different times, the magnetic field meter 403 or its equivalent may be used in the identical position relative to the magnetized object 402 to measure the magnetic field strength. If the strength of the field has faded below a certain level, the patient may receive a treatment to recharge the object.

FIG. 8 illustrates a pill 500. The pill 500 has metallic particles 502, such as ferrous particles, that are microencapsulated or encapsulated in some form. The magnetizing devices of the present invention are used to magnetize the pill 500 and then the pill 500 is swallowed. A meter can be used to measure the magnetic field emanating from the pill 500 before it is swallowed or after it is swallowed. Such a magnetized pill can provide therapeutic magnetic field therapy to digestive systems in treatment of such situations as irritable bowel syndrome.

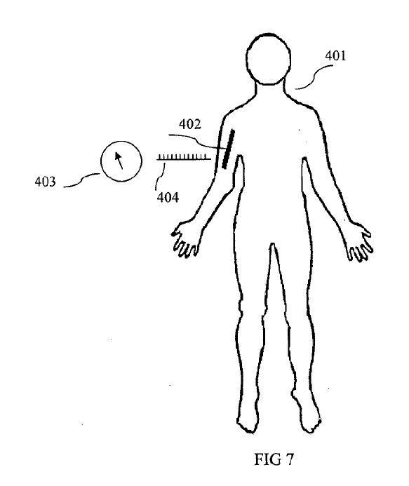

FIG. 9 illustrates a kit. The kit includes a magnetic field generator 602 and a meter 600, both of which are enclosed in a single package 604.

The magnetic therapies described herein, are useful in promoting the general wellness of persons. They are also useful in promoting the general wellness of animals.

The use of a meter creates repeatable and measurable therapeutic magnetic treatments. It is also important in clinical studies to correlate the healing of the patient and pain relief experiences with the intensity of the magnetism to develop the optimum treatment for each stage. By combining the machine that generates the magnetism with a reader that measures the magnetism of the metal part or micro encapsulated product the present invention is of significant medical use in the control of pain and in the healing of certain types of medical problems.

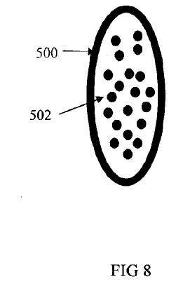

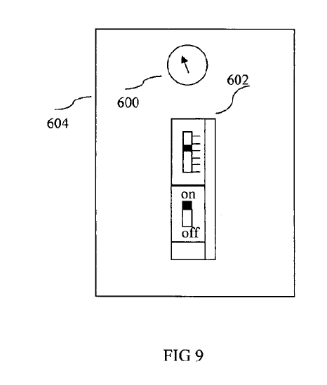

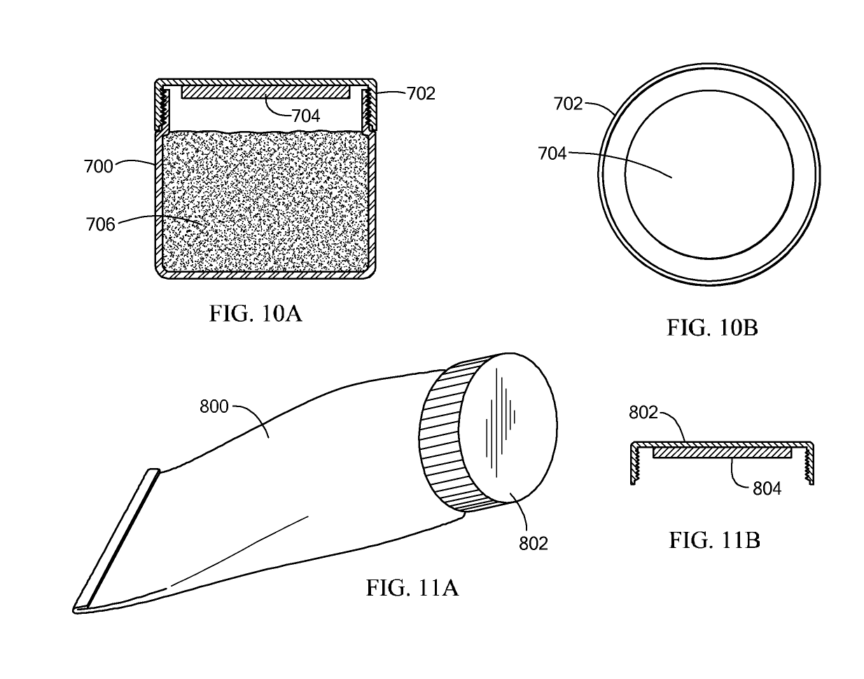

Magnetizable material for use in these therapies may be advantageously provided in a topical preparation for application to the skin. Initial magnetization of the material in accordance with embodiments of the present invention, may be performed by a magnetic source in the container of the preparation. For example, in FIG. 10A, the container is a jar 700. The cap 702 on the jar includes a permanent magnet 704 as shown in FIGS. 10A and 10B). The magnet 704 induces a magnetic field in the magnetizable material mixed into the topical preparation. The magnetizable material is preferably magnetizable particles, such as metal or ferrous particles. The particles may be microencapsulated.

The topical preparations 706 may be any conventionally used carrier such as cream, ointment, gel or the like suitable for application to a patient's skin. It is preferred, however, that the preparation have a viscosity sufficiently high to prevent the magnet 704 from causing translational movement of the magnetizable particles through its preparation. It is desirable to maintain a relatively homogenous mixture with the particles relatively evenly mixed throughout the preparation in the container. The particles are preferably relatively separated and the magnetic source should not induce clumping.

In an alternative embodiment, the container may be a tube 800 with a cap 802 carrying the magnetic source. A permanent magnet 804 is shown in the cap 802 in FIGS. 11A and 11B.

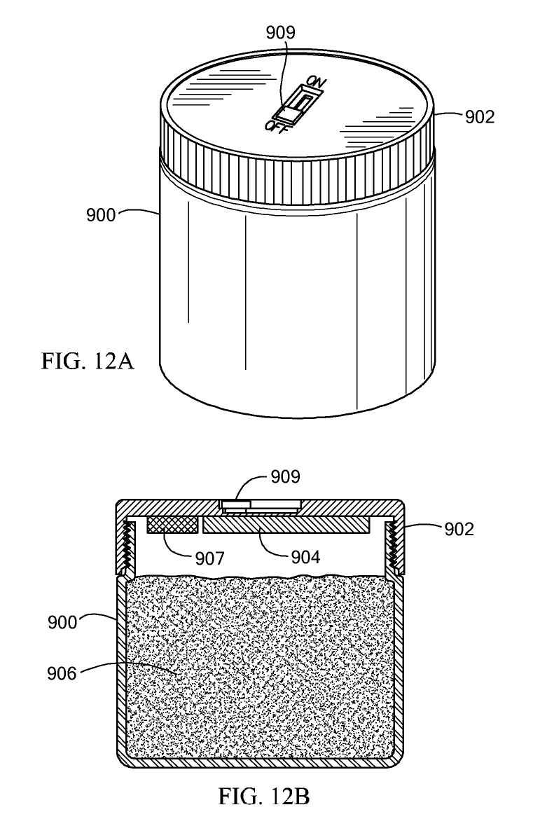

While the containers described above have permanent magnets associated therewith, in an alternate embodiment exemplified by FIGS. 12 A and 12B, the magnetic source may be an electromagnet 904. The electromagnetic coil 904 may be connected to a battery 907, also housed by the container 900. To extend the life of the battery, it would be advantageous to include an electronic switch to disable current from the battery to the electromagnet 904 when the preparation is not being used for an extended time. A circuit is created through the battery 907, the electromagnet 904 and the electrical switch when the switch is on to enable current flow. The electrical switch may be operated by an activator, such as a button or a slide switch 909, on the outside of the cap or container body. Alternatively, one might choose to locate the switch activator on the underside of the cap to reduce unintended activation. As a further feature, an indicator light such as an LED might be included on the outside of the container or its cap to inform as to whether the current is flowing to the electromagnet 904.

While three examples of containers are illustrated herein, other embodiments of the present invention include other containers for holding topical preparations. Although including the magnetic source in the cap is preferred, one could alternatively line portions of the container with a magnetic source to apply a magnetic field to the preparation within the container. One advantage of putting the magnet in the cap is that it is closest to the preparation that will be used next.

By using a topical preparation with magnetizable material, one can spread the preparation on specific areas of the body where magnetic therapy would be useful. The magnetic field generator and meter can be used to maintain a desired level of magnetism on the skin to achieve the desired treatment. Advantageously, the need for a magnetic field generator can be avoided by making use of the container itself to recharge the magnetic field of the magnetizable material on a patient's skin. The container or its cap only need be brought near the skin on which the topical preparation was applied. By bringing the magnetic source near the magnetizable material for a period of time, its magnetic field may thus be adequately recharged.

Modifications of this invention will be readily apparent to those skilled in the art and it is intended that the invention be not limited by the embodiments disclosed herein but that the scope of the invention be defined by the appended claims.

I claim:

1. A packaged topical preparation for use in therapeutic magnetic therapies, comprising:
a container;
a topical preparation mixed with magnetizable material and stored within the container; and
a magnetic source attached to the container.

2. The packaged topical preparation of claim 1, wherein the container further comprises a cap and wherein the magnetic source is mounted in the cap.

3. The packaged topical preparation of claim 1, wherein the topical preparation has a viscosity sufficient to prevent the magnetizable material from translational movement responsive to the magnetic source.

4. The packaged topical preparation of claim 3, wherein the topical preparation is a cream.

5. The packaged topical preparation of claim 3, wherein the topical preparation is a gel.

6. The packaged topical preparation of claim 3, wherein the topical preparation is an ointment.

7. The packaged topical preparation of claim 1, wherein the container comprises a jar.

8. The packaged topical preparation of claim 1, wherein the container comprises a tube.

9. The packaged topical preparation of claim 1, wherein the magnetizable material comprises micro-encapulated ferrous particles.

10. The packaged topical preparation of claim 1, wherein the magnetic source comprises a permanent magnet.

11. The packaged topical preparation of claim 1, wherein the magnetic source comprises an electromagnet and further comprising a battery electrically coupled to the electromagnet.

12. The packaged topical preparation of claim 11, further comprising a switch for enabling or disabling current from the battery into the electromagnet.

13. A packaged topical preparation for use in therapeutic magnetic therapies comprising:
a container having a cap;
a magnetic source in the cap; and
a topical preparation mixed with magnetic material and stored within the container, the preparation having a viscosity sufficient to prevent the magnetizable material from translational movement responsive to the magnetic source.

14. The packaged topical preparation of claim 13 wherein the topical preparation is a cream.

15. The packaged topical preparation of claim 13 wherein the topical preparation is a gel.

16. The packaged topical preparation of claim 13 wherein the topical preparation is an ointment.

17. The packaged topical preparation of claim 13 wherein the container comprises a jar.

18. The packaged topical preparation of claim 13 wherein the container comprises a tube.

19. The packaged topical preparation of claim 13 wherein the magnetizable material comprises micro-encapulated ferrous particles.

20. The packaged topical preparation of claim 13, wherein the magnetic source comprises a permanent magnet.

21. The packaged topical preparation of claim 13, wherein the magnetic source comprises an electromagnet and further comprising a battery electrically coupled to the electromagnet.

22. The packaged topical preparation of claim 21, further comprising a switch for enabling or disabling current from the battery into the electromagnet.

23. A method of providing a magnetic field comprising:
providing a container of topical preparation mixed with magnetizable particles, wherein the container includes a magnetic source;
applying a portion of the topical preparation on the skin of a patient; and
recharging the magnetic field produced by the magnetized particles by bringing the container near the skin for a period of time.
